(19) United States
(12) Patent Application Publication
(10) Pub. No.: US 2016/0331896 A1
(43) Pub. Date: Nov. 17, 2016

(54) IMAGING SYSTEM AND INJECTING APPARATUS

(71) Applicant: NEMOTO KYORINDO CO., LTD., Tokyo (JP)

(72) Inventors: Shigeru NEMOTO, Tokyo (JP); Tomoyuki ZAMAMI, Tokyo (JP); Yumiko FUKIKOSHI, Tokyo (JP); Tomoyuki ARAKI, Tokyo (JP); Hitoshi SUGAWARA, Tokyo (JP)

(73) Assignee: NEMOTO KYORINDO CO., LTD., Tokyo (JP)

(21) Appl. No.: 15/111,699

(22) PCT Filed: Jan. 20, 2015
(86) PCT No.: PCT/JP2015/000221

§ 371 Date: Jul. 14, 2016

(30) Foreign Application Priority Data
Jan. 22, 2014 (JP) 2014-009094

(51) Int. Cl. A61M 5/172; A61M 5/00; A61B 6/00; A61B 8/08; A61B 5/055; A61B 6/03

(52) U.S. Cl. CPC A61M 5/1723; A61B 5/055; A61B 6/032; A61B 6/037; A61B 6/504; A61B 8/481; A61M 5/007

(57) ABSTRACT

An imaging system capable of setting an optimum injection condition in accordance with an imaging condition and preventing erroneous inputs and erroneous operations when determining the injection condition based on the imaging condition. The imaging system includes an injecting apparatus for a chemical liquid and an imaging apparatus, which is connectable to the injecting apparatus. The imaging apparatus includes: a control unit to determine a first imaging condition based on externally acquired object information, and to transmit the first imaging condition to the injecting apparatus; and an input unit to input a second imaging condition changed from the first imaging condition. The injecting apparatus includes a control unit to determine a first injection condition based on the first imaging condition, and to change, when the second imaging condition is received from the imaging apparatus, the first injection condition to a second injection condition based on the second imaging condition.

### TECHNICAL FIELD

The present invention relates to an imaging system for medical use, and more particularly, to an imaging system including an imaging apparatus for medical use and an injecting apparatus of a chemical liquid, and to an injecting apparatus of this imaging system.

### BACKGROUND ART

As an imaging system for medical use, in Patent Literature 1, there is a description of an imaging system capable of performing, by a shared operation device, data input for setting an injection condition for injecting a chemical liquid by a chemical liquid injecting apparatus and data input for setting an imaging condition for taking an image by a computed tomography (CT) scanner.

### CITATION LIST

### Patent Literature

PTL 1: WO 2008/018385 A1

### SUMMARY OF INVENTION

### Technical Problem

However, in the system of Patent Literature 1, the injection condition is determined based on data input from the shared operation device. As a result, when changing the injection condition of the chemical liquid based on the set imaging condition, data needs to be input again by using the operation device. Further, the injection condition needs to be changed after the imaging condition has been verified by the operator.

### Solution to Problem

In order to solve the above-mentioned problems, according to one embodiment of the present invention, there is provided an imaging system, including an injecting apparatus for a chemical liquid and an imaging apparatus for medical use, which is connectable to the injecting apparatus and which takes an image of an object, the imaging apparatus including: a control unit configured to determine a first imaging condition based on externally acquired object information, and to transmit the first imaging condition to the injecting apparatus; and an input unit configured to input a second imaging condition, which is changed from the first imaging condition, the injecting apparatus including: a control unit configured to determine a first injection condition based on the first imaging condition, and to change, when the second imaging condition is received from the imaging apparatus, the first injection condition to a second injection condition based on the second imaging condition.

Further, according to another embodiment of the present invention, there is provided an imaging system, including an injecting apparatus for a chemical liquid and an imaging apparatus for medical use, which is connectable to the injecting apparatus and which takes an image of an object, the imaging apparatus including: a control unit configured to determine a first imaging condition based on externally acquired object information, and to transmit the first imaging condition to the injecting apparatus; and an input unit configured to input a second imaging condition, which is changed from the first imaging condition, the injecting apparatus including: a control unit configured to determine a first injection condition based on the first imaging condition, and to change, when the second imaging condition is received from the imaging apparatus, the first injection condition to a second injection condition based on the second imaging condition, wherein the control unit of the imaging apparatus is configured to: acquire a third injection condition, externally; compare the first injection condition or the second injection condition with the third injection condition; and perform a mismatch notification when the first injection condition or the second injection condition mismatches with the third injection condition.

Further, according to another embodiment of the present invention, there is provided for a chemical liquid, which is connectable to an imaging apparatus for medical use, which takes an image of an object, the injecting apparatus including: a control unit configured to determine a first injection condition based on a first imaging condition received from the imaging apparatus, and to change, when a second imaging condition which is changed from the first imaging condition is received from the imaging apparatus, the first injection condition to a second injection condition based on the second imaging condition.

As a result, the setting of the optimum injection condition in accordance with the imaging condition can be easily performed. Further, erroneous inputs and erroneous operations can be prevented when determining the injection condition based on the imaging condition.

Further features of the present invention will become apparent from the following description of exemplary embodiments referring to the attached drawings.

### BRIEF DESCRIPTION OF DRAWINGS

FIG. 1 is a schematic diagram for illustrating an imaging system according to a first embodiment of the present invention.

FIG. 2 is a schematic block diagram for illustrating the imaging system according to the first embodiment.

FIG. 3 explains setting of an imaging condition and an injection condition in the imaging system according to the first embodiment.

FIG. 4 explains setting of an imaging condition and an injection condition in an imaging system according to a second embodiment of the present invention.

FIG. 5 explains setting of an imaging condition and an injection condition in an imaging system according to a modified embodiment of the present invention.

### DESCRIPTION OF EMBODIMENTS

Exemplary embodiments for carrying out the present invention are described in detail below with reference to the drawings. Note that, dimensions, materials, shapes, the relative positions of components, and the like that are mentioned in the following embodiments can be changed arbitrarily to suit the structure of a device to which the present invention is applied, or various conditions. The scope of the present invention is not limited to the embodiments described concretely in the following, unless otherwise specially noted.

### First Embodiment

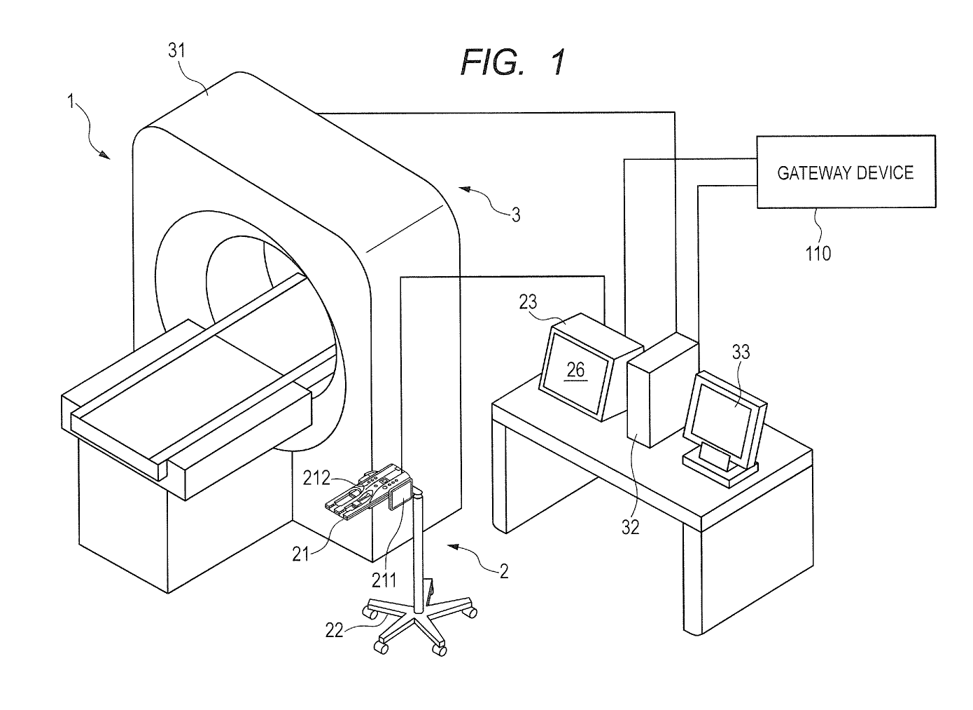

As illustrated in FIG. 1, an imaging system 1 includes an injecting apparatus 2 of a chemical liquid, and an imaging apparatus 3 for medical use, which is connected to the injecting apparatus 2 and is configured to take an image of an object. The injecting apparatus 2 and the imaging apparatus 3 are connected by a cable or wirelessly via a gateway device 110. The injecting apparatus 2 is configured to inject a chemical liquid filled into a syringe, e.g., physiological saline or various contrast media, into the body of a patient serving as the object. Examples of the imaging apparatus 3 include various medical imaging apparatus, such as a magnetic resonance imaging (MRI) apparatus, a CT apparatus, an angiographic imaging apparatus, a positron emission tomography (PET) apparatus, a single photon emission computed tomography (SPECT) apparatus, a CT angiographic apparatus, an MR angiographic apparatus, an ultrasonic diagnostic apparatus, and a vascular imaging apparatus. In the first embodiment, the imaging system 1 includes a CT apparatus as the imaging apparatus 3.

The injecting apparatus 2 includes an injection head 21, which serves as an injection unit, to which a syringe filled with the chemical liquid is mounted, a stand 22 configured to hold the injection head 21, and a console 23, which serves as a control device, connected to the injection head 21. The injection head 21, which is configured to inject the chemical liquid, includes a display 211. The injecting apparatus 2 may include, in place of the console 23 as a control device, a control device connected to the injection head 21 and a display unit (touch panel display, etc.), which is connected to the control device and which is configured to display, for example, an injection state of the chemical liquid. In this case, the injection head 21 and the control device may be integrally formed with the stand 22. Further, a ceiling member may be arranged in place of the stand 22, and the injection head 21 may be hung from the ceiling via the ceiling member.

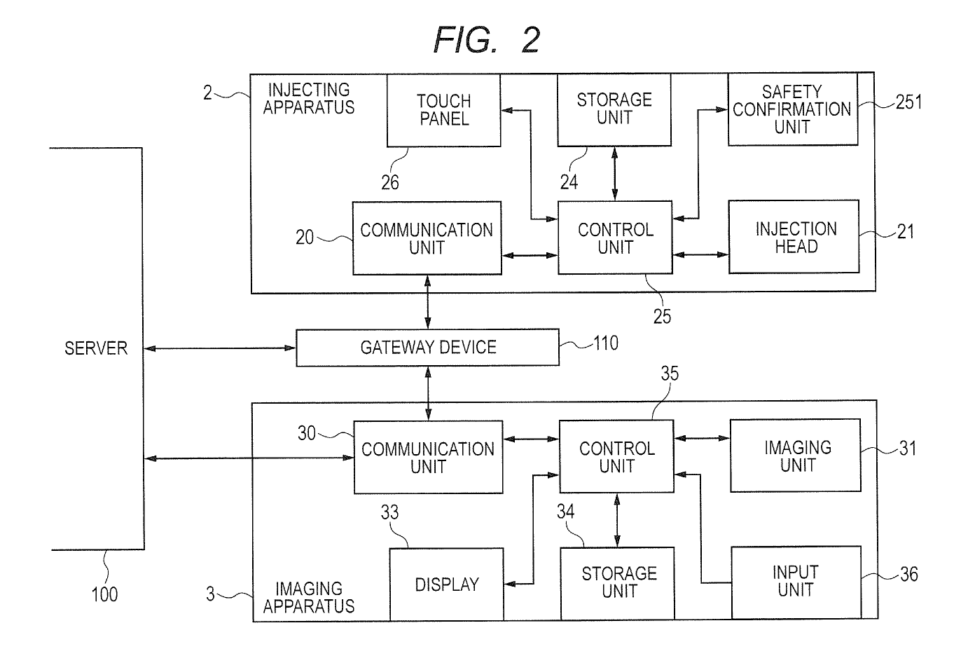

The console 23, which includes a communication unit 20 (FIG. 2), may perform communication to and from the injection head 21, the imaging apparatus 3, the gateway device 110, the server 100, and the like, via the communication unit 20 by a cable or wirelessly. Further, the injecting apparatus 2 may include, for example, a power supply or a battery, a hand switch connected to the console 23, or a remote operation device, which is configured to remotely operate the injection head 21. The remote operation device is capable of remotely operating the injection head 21 to start or stop the injection. Further, the power supply or the battery may be arranged in any one of the injection head 21 and the control device (console 23), or may be arranged separately from the injection head 21 and the control device (console 23).

The imaging apparatus 3 includes an imaging unit 31 configured to expose the patient to X-rays, a control device 32 connected to the imaging unit 31, and a display 33, which serves as a display unit, connected to the control device 32. The control device 32 and the display 33 of the imaging apparatus 3 may be integrally formed.

The imaging unit 31 includes, for example, a bed, an X-ray source configured to irradiate the patient, which serves as the object, with X-rays, and an X-ray detector configured to detect X-rays that have passed through the patient. Further, the imaging unit 31 is configured to take a fluoroscopic image of the patient by exposing the patient to X-rays and back-projecting the internal structures of the patient based on the X-rays that have passed through the patient. The imaging unit 31 may also be configured to take an image with use of radio waves or ultrasonic waves in place of X-rays. The control device 32, which includes a communication unit 30 (FIG. 2), may perform communication to and from the imaging unit 31, the injecting apparatus 2, the gateway device 110, the server 100, and the like, via the communication unit 30 by a cable or wirelessly.

The injection head 21 includes a syringe holding unit to which the syringe is mounted, and a drive mechanism configured to push out the chemical liquid in the syringe based on a determined injection protocol. The injection protocol contains information on an injection condition, e.g., an injection amount, an injection rate, an injection time, an injection timing, a contrast medium concentration, and an injection pressure. Further, the injection head 21 includes the head display 211, which is configured to display the injection condition, the injection state, a device input state, a setting state, various injection results, and the like, and an operation unit 212 for inputting an operation of the drive mechanism.

The operation unit 212 includes a forward button for the drive mechanism, a back button for the drive mechanism, a final confirmation button, and the like. When injecting the chemical liquid, an extension tube is connected to a tip portion of the syringe mounted to the injection head 21. When injection preparation, e.g., connecting the extension tube, is complete, the operator presses the final confirmation button. As a result, the injection head 21 is in a state ready to start the injection. The chemical liquid pushed out from the syringe is injected into the body of the patient via the extension tube and the like.

Various types of syringe, e.g., a pre-filled syringe having a radio-frequency identification (RFID) chip, may be mounted to the injection head 21. Further, the injection head 21 includes a reading unit configured to read the RFID chip mounted on the syringe. The RFID chip is configured to store chemical liquid information relating to the chemical liquid. Examples of the chemical liquid information include a product name, a product ID, a chemical classification, a contained component, a concentration, a viscosity, an expiration date, a syringe volume, a syringe pressure resistance, a syringe inner diameter, a piston stroke, a lot number, and the like.

Examples of the server 100 (external storage device) illustrated in FIG. 2 include a radiology information system (RIS), a picture archiving and communication system (PACS), a hospital information system (HIS), and the like. Further, an image detection system, an image creation workstation, or the like, may also be used. The imaging apparatus 3 is capable of receiving information from the server 100 via the communication unit 30. Further, the imaging apparatus 3 is capable of transmitting information to the server 100 via the communication unit 30. The injecting apparatus 2 is capable of receiving information from the server 100 via the imaging apparatus 3, the gateway device 110, and the communication unit 20. In addition, the injecting apparatus 2 is capable of transmitting information to the server 100 via the communication unit 20, the gateway device 110, and the imaging apparatus 3.

An examination order is stored in advance in the server 100. The examination order contains object information as information relating to the patient, and examination information as information relating to the examination content. Examples of the object information may include a patient number (patient ID), a patient name, a sex, a date of birth, an age, a height, a weight, a blood volume, a body surface area, a patient illness, a heart rate, a cardiac output, and the like. Examples of the examination information may include an examination number (examination ID), an examination site (imaging site), an examination date and time, a chemical liquid type, a chemical liquid name, an imaging plan, and the like. The server 100 is capable of storing information relating to an imaging result, such as data of an image transmitted from the imaging apparatus 3, and information relating to an injection result transmitted from the injecting apparatus 2.

The gateway device 110 connected to the injecting apparatus 2 and the imaging apparatus 3 is also connected to the server 100. The gateway device 110 is capable of converting data received from the imaging apparatus 3, and transmitting the converted data to the injecting apparatus 2. Similarly, the gateway device 110 is capable of converting data received from the injecting apparatus 2, and transmitting the converted data to the imaging apparatus 3. Further, the gateway device 110 is capable of converting data received from the imaging apparatus 3 and data received from the injecting apparatus 2, and transmitting the converted data to the server 100. The injecting apparatus 2, the imaging apparatus 3, and the server 100 are configured to transmit and receive various kinds of data to and from each other based on a medical data standard, e.g., Digital Imaging and Communications in Medicine (DICOM) and Health Level Seven (HL7). The injecting apparatus 2 and the server 100 may also be connected via the communication unit 20, and be configured to directly transmit and receive data without going via the imaging apparatus 3.

As illustrated in FIG. 2, the imaging apparatus 3 includes, as a display unit, the display 33 on which the imaging plan and the like are displayed, as a storage unit 34, a memory configured to store a control program and information relating to the imaging plan, for example, and as a control unit 35, a central processing unit (CPU) configured to control the imaging apparatus 3. The imaging apparatus 3 also includes the imaging unit 31, which is configured to take an image of the patient, and as an input unit 36, a keyboard and the like to be used by the operator to input information. The imaging plan contains information relating to the imaging condition, e.g., the imaging site, an effective tube voltage, a model name, a manufacturer name, the imaging time, a tube voltage, an imaging range, a rotating speed, a helical pitch, an exposure time, a radiation dose, and an imaging method.

The control unit 35 is configured to determine a first imaging condition, which is a base imaging condition, based on the object information contained in the examination order acquired from an external device, e.g., the server 100, and to store the determined first imaging condition in the storage unit 34. Further, the control unit 35 is configured to transmit the first imaging condition to the injecting apparatus 2 via the communication unit 30. The operator can input a second imaging condition, which is the imaging condition after a change has been made to the first imaging condition, to the control unit 35 via the input unit 36. The control unit 35 is configured to store the second imaging condition in the storage unit 34.

The control unit 35 is configured to control the whole imaging apparatus 3 based on, for example, a control program stored in the storage unit 34. Further, the control unit 35 is also configured to comprehensively control various processes such as calculations, controls, and determinations. In other words, the control unit 35 is configured to determine the imaging condition of the imaging plan based on, for example, information acquired from an external device, e.g., the server 100, information stored in the storage unit 34, and information input from the input unit 36. In addition, the control unit 35 controls the imaging unit 31 to take an image of the patient according to the determined imaging plan. In the first embodiment, the storage unit 34 and the control unit 35 are arranged in the control device 32 of the imaging apparatus 3, and the control device 32 is connected to the display 33. The display 33 is configured to display an input state of the imaging apparatus 3, a setting state of the imaging apparatus 3, an imaging result, various types of information, and the like.

The injecting apparatus 2 includes, as a storage unit 24, a memory configured to store a control program, information relating to the injection protocol, information relating to the chemical liquid, and the like, as a control unit 25, a CPU configured to control the injecting apparatus 2, as a display/input unit, a touch panel 26 configured to display the injection protocol and the like as well as function as an input unit, and the injection head 21, which is configured to inject the chemical liquid. In the first embodiment, the touch panel 26, the storage unit 24, and the control unit 25 are arranged in the console 23. In addition to the injection protocol, the touch panel 26 is configured to display an input state of the injecting apparatus 2, a setting state of the injecting apparatus 2, an injection result, various types of information, and the like. The operator can input a predetermined command, data, or the like, via the touch panel 26. The injecting apparatus 2 may include, in place of the touch panel 26, a display as the display unit and a user interface, e.g., a keyboard, as the input unit.

The control unit 25 is configured to receive the first imaging condition from the imaging apparatus 3 via the communication unit 20, and to determine a first injection condition, which is an injection condition reflecting the initial imaging condition, based on the first imaging condition. Further, the control unit 25 is configured to determine, when the second imaging condition has been received from the imaging apparatus, a second injection condition based on the second imaging condition, and to change the first injection condition to the second injection condition.

The control unit 25 is configured to control the whole injecting apparatus 2 based on a control program and the like stored in the storage unit 24. Further, the control unit 25 is also configured to comprehensively control various processes such as calculations, controls, determinations. In other words, the control unit 25 is configured to determine the injection condition of the injection protocol based on, for example, information acquired from an external device, e.g., the imaging apparatus 3, information stored in the storage unit 24, and information input from the touch panel 26. The control unit 25 is also configured to then inject the chemical liquid by controlling the injection head 21 such that the injection head 21 follows the determined injection protocol.

The storage unit 24 of the injecting apparatus 2 and the storage unit 34 of the imaging apparatus 3 each include a random-access memory (RAM), which is a system work memory for operating the control unit, a read-only memory (ROM) configured to store programs, system software, and the like, or a hard disk drive. Further, the control unit 25 of the injecting apparatus 2 and the control unit 35 of the imaging apparatus 3 are each configured to control various processes based on programs stored on a portable recording medium, e.g., a compact disc (CD) and a digital versatile disc (DVD), or an external storage medium, e.g., a server on the Internet.

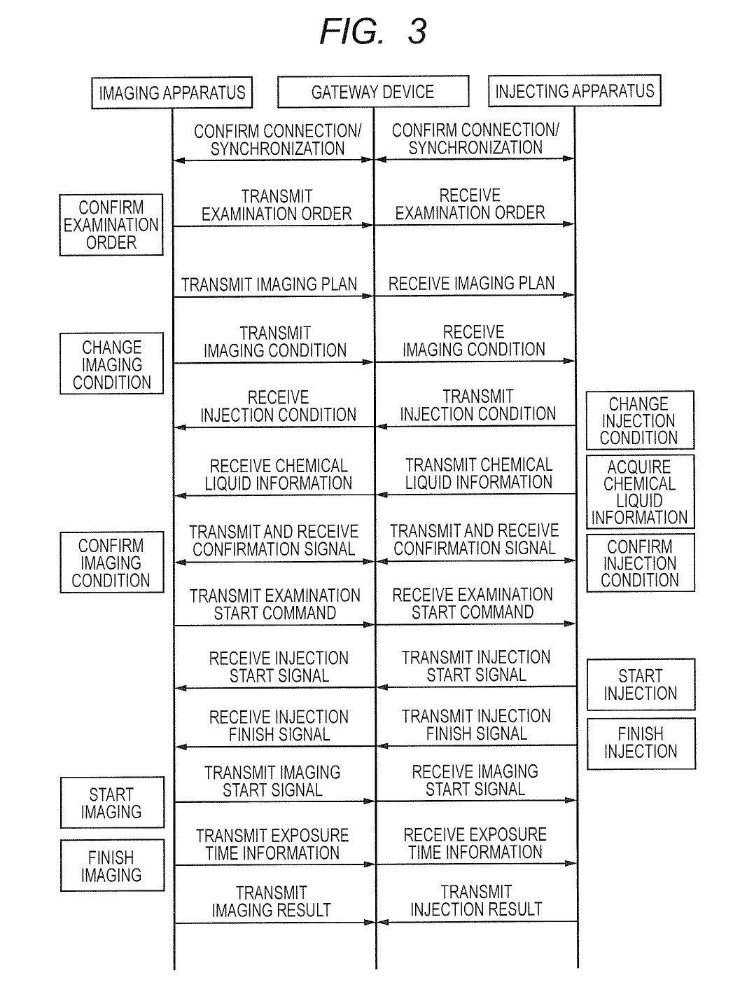

Next, imaging according to the first embodiment is described with reference to FIG. 3. The various processes performed by the imaging apparatus 3 in the following description are each controlled by the control unit 35 of the imaging apparatus 3 based on a control program. Similarly, the various processes performed by the injecting apparatus 2 are each controlled by the control unit 25 of the injecting apparatus 2 based on a control program.

When the operator turns on the power supply of the imaging apparatus 3 and the power supply of the injecting apparatus 2, the imaging apparatus 3 and the injecting apparatus 2 perform a connection/synchronization confirmation with each other. The imaging apparatus 3 acquires an examination order including object information from the external server 100, and displays the acquired examination order on the display 33. The operator then verifies the content of the displayed examination order. In the case that the imaging apparatus 3 has acquired a plurality of examination orders, the operator verifies the content of the examination orders, and selects a desired examination order. The imaging apparatus 3 may also display a status or a confirmation result of the connection/synchronization confirmation on the display 33. Further, the injecting apparatus 2 may display the status or the confirmation result of the connection/synchronization confirmation on the touch panel 26 or the head display 211. As a result, when the injecting apparatus 2 and the imaging apparatus 3 are not working together because connection/synchronization has not been established, erroneous operations by the operator can be prevented, and hence safety is improved.

The imaging apparatus 3 acquires the object information from the examination order, and transmits the examination order, which has been verified by the operator, to the injecting apparatus 2 via the gateway device 110. This enables the injecting apparatus 2 to acquire the examination order stored in the server 100 via the imaging apparatus 3. The injecting apparatus 2 then acquires from the examination order the object information, e.g., the weight of the patient, which is required in order to determine the injection protocol. Further, the injecting apparatus 2 determines a preliminary injection condition, which is an injection condition based on the examination order. In other words, the injecting apparatus 2 determines the preliminary injection condition based on the object information acquired from the examination order. For example, the injecting apparatus 2 performs calculations and the like for determining the injection amount of the chemical liquid based on the weight of the patient. Then, the injecting apparatus 2 displays the determined injection condition on the touch panel 26.

The injecting apparatus 2 may also calculate, based on a standard imaging condition stored in advance in the storage unit 24, the injection condition in accordance with the standard imaging condition before acquiring the imaging plan or the imaging condition. In this case, the field on the touch panel 26 in which the injection condition to be calculated after the imaging plan or the imaging condition has been acquired is displayed is no longer blank. In other words, the injecting apparatus 2 displays, even for an injection condition to be calculated after the imaging plan or the imaging condition has been acquired, the injection condition based on the standard imaging condition on the touch panel 26. As a result, the operator can compare and verify the injection condition based on the standard imaging condition and the injection condition based on the imaging condition of the determined imaging plan.

The imaging apparatus 3 determines the imaging plan including the first imaging condition in accordance with the examination order, and displays the determined imaging plan on the display 33. As a result, the operator can verify the content of the displayed imaging plan. At this point, the imaging apparatus 3 is capable of determining a plurality of imaging plans in accordance with the examination order. In this case, the operator verifies the content of the imaging plans, and selects a desired imaging plan. Then, the imaging apparatus 3 transmits the imaging plan verified by the operator to the injecting apparatus 2, and the injecting apparatus 2 receives the imaging plan.

When the injecting apparatus 2 receives the imaging plan including the first imaging condition, the injecting apparatus 2 reflects the imaging plan in the injection protocol. In other words, the injecting apparatus 2 acquires from the received imaging plan the first imaging condition, for example, the imaging time and the like, which is required for determining the injection protocol. Then, the injecting apparatus 2 automatically changes the preliminary injection condition based on the first imaging condition. For example, the injecting apparatus 2 lengthens or shortens the injection time based on the imaging time. However, when it is not necessary to change the preliminary injection condition, the injection protocol is maintained without being changed.

In other words, the imaging apparatus 3 transmits, before transmission of the first imaging condition, the examination order including the object information to the injecting apparatus 2. Then, the injecting apparatus 2 acquires the object information from the examination order, and determines the preliminary injection condition based on the acquired object information. When the injecting apparatus 2 has received the first imaging condition from the imaging apparatus 3, the injecting apparatus 2 changes the preliminary injection condition to the first injection condition based on the first imaging condition.

After the operator has verified and selected the imaging plan, if there is a defect in the first imaging condition, the operator changes the first imaging condition to the second imaging condition via the input unit 36 of the imaging apparatus 3. In this case, the imaging apparatus 3 transmits the second imaging condition obtained by the change to the injecting apparatus 2. When the operator has changed the first imaging condition a plurality of times, the imaging apparatus 3 transmits the second imaging condition to the injecting apparatus 2 each time the first imaging condition is changed. The injecting apparatus 2 automatically changes the first injection condition so as to become the injection protocol based on the acquired second imaging condition. For example, based on the changed tube voltage, the injecting apparatus 2 increases or decreases the contrast medium amount, an iodine content, or a contrast medium concentration. However, when it is not necessary to change the first injection condition, the injection protocol is maintained without being changed.

As a result, the injecting apparatus 2 is capable of determining the optimum injection protocol by the operator simply selecting the imaging plan and changing the imaging condition as necessary. Consequently, tasks for the operator such as inputting the required information into the injecting apparatus 2 and changing the injection condition based on the imaging condition can be omitted. Further, erroneous inputs and erroneous operations due to those tasks can be prevented.

When determining the injection protocol or changing the injection condition, the injecting apparatus 2 determines the injection protocol such that the injection condition satisfies a chemical liquid injectable condition, which is stored in advance in the storage unit 24. In other words, the injecting apparatus 2 determines the preliminary injection condition, the first injection condition, and the second injection condition such that each of those injection conditions satisfies the injectable condition. For example, the injecting apparatus 2 calculates the injection condition within a range that does not exceed an injectable range determined in advance.

The injecting apparatus 2 includes a safety confirmation unit 251 configured to determine, when the injection condition has been acquired from an external device, e.g., the imaging apparatus 3, whether or not the acquired injection condition satisfies the injectable condition. When the injecting apparatus 2 has acquired the injection condition manually input or changed by the operator, the injecting apparatus 2 causes the safety confirmation unit 251 to compare the acquired injection condition with the injectable condition. When it is determined by the safety confirmation unit 251 that the injectable condition is not satisfied, the injecting apparatus 2 automatically changes the injection condition such that the injection condition satisfies the injectable condition. The injecting apparatus 2 may display on the touch panel 26 the injection conditions before and after the change and an indication that the injection condition has been automatically changed. As a result, the operator can compare and verify the input injection condition and the automatically changed injection condition.

For example, when the injection rate is higher than an injectable rate by the injection head 21, the injecting apparatus 2 automatically changes the injection condition such that the injection rate of the injection protocol is decreased. When the patient is an infant, and the injection amount of the chemical liquid exceeds the injectable amount for infants, the injecting apparatus 2 automatically changes the injection condition such that the injection amount of the injection protocol is decreased. When the injection time is shorter than the imaging time, the injecting apparatus 2 automatically changes the injection condition such that the injection time is increased. In the case that the injection condition does not satisfy the injectable condition, the injecting apparatus 2 may display a warning on the touch panel 26. In this case, the injecting apparatus 2 does not start the injection of the chemical liquid even when an instruction to start the injection has been issued. As a result, injection of the chemical liquid based on an erroneous injection condition can be reliably prevented.

When the injection condition has been determined and when the injection condition has been changed, the injecting apparatus 2 transmits those injection conditions to the imaging apparatus 3, and displays the injection conditions on the touch panel 26. The imaging apparatus 3, which has received those injection conditions, may display the received injection conditions on the display 33. As a result, the operator can verify the content of the injection protocol on any one of the screen of the touch panel 26 of the injecting apparatus 2 and the screen of the display 33 of the imaging apparatus 3.

The injecting apparatus 2 may also transmit the information displayed on the touch panel 26 to the injection head 21, and display the transmitted information on the head display 211. Specifically, the injecting apparatus 2 may display each injection condition on the head display 211 of the injection head 21 at the same time that the display unit (touch panel 26) of the console 23 displays the preliminary injection condition, the first injection condition, or the second injection condition. Further, the injecting apparatus 2 may also display the first imaging condition and the second imaging condition on the head display 211 at the same time that the display unit of the console 23 displays the received first imaging condition and second imaging condition of the imaging plan. For example, the injecting apparatus 2 may display an exposure start (expected) timing or an imaging start timing, for example, on the head display 211. As a result, the operator, who is near the injecting apparatus 2, can verify the injection protocol, the imaging plan, or both the injection protocol and the imaging plan. Further, usually, the injection head 21 is arranged near the patient. As a result, the operator can verify the injection protocol, the imaging plan, or both the injection protocol and the imaging plan, while looking at the condition of the patient.

The injecting apparatus 2 may also display, simultaneously with each of the injection conditions, information, such as the patient name, the patient ID, or the examination ID, on the head display 211. As a result, the chemical liquid can be prevented from being injected into the wrong patient, and hence safety is improved.

When it is necessary, as a result of verification by the operator, for the injection condition to be changed because the state of the patient has changed, for example, the operator manually changes the injection condition via the touch panel 26. In this case, the injecting apparatus 2 performs a safety confirmation of the input injection condition by the safety confirmation unit 251. When the injectable condition is satisfied, the input injection condition is transmitted to the imaging apparatus 3. The imaging apparatus 3 may display the received injection condition on the display 33.

The reading unit of the injection head 21 acquires, when a syringe having an RFID chip is mounted, the chemical liquid information from the RFID chip. Then, the injecting apparatus 2 transmits the chemical liquid information to the imaging apparatus 3. At the same time, the injecting apparatus 2 displays the chemical liquid information on the touch panel 26. As a result, the operator can verify whether or not the correct syringe is mounted at the same time as verifying the injection protocol. Further, the imaging apparatus 3, which has received the chemical liquid information, may also display the chemical liquid information on the display 33. As a result, the operator can verify the content of the chemical liquid information on any one of the screen of the touch panel 26 of the injecting apparatus 2 and the screen of the display 33 of the imaging apparatus 3. Even when the syringe has been mounted before determining the injection protocol, the injecting apparatus 2 transmits the chemical liquid information to the imaging apparatus 3 when the chemical liquid information has been acquired.

When the imaging condition has been verified by the operator and the imaging preparation is complete, the operator presses a confirmation button displayed on the display 33 by operating the input unit 36 of the imaging apparatus 3. Then, the imaging apparatus 3 transmits a confirmation signal to the injecting apparatus 2, and waits in a state ready to start the imaging. On the other hand, when the injection condition has been verified by the operator and the injection preparation is complete, the operator presses a confirmation button displayed on the touch panel 26 of the injecting apparatus 2. As a result, the injecting apparatus 2 transmits a confirmation signal to the injecting apparatus 2, and waits in a state ready to start the injection. The injecting apparatus 2 may be configured to not display the confirmation button on the touch panel 26 when the final confirmation button of the injection head 21 has not been pressed. It does not matter whether the imaging preparation or the injection preparation is completed first.

The imaging preparation and the injection preparation are complete when the imaging apparatus 3 receives the confirmation signal from the injecting apparatus 2 and the injecting apparatus 2 receives the confirmation signal from the imaging apparatus 3. When the injection and the imaging are capable of starting, the injecting apparatus 2 and the imaging apparatus 3 may visually or aurally indicate to the operator to start the examination. For example, the injecting apparatus 2 and the imaging apparatus 3 may display an indication representing the examination start or generate a sound representing the examination start. Then, the operator presses a start button arranged on the imaging apparatus 3. As a result, the imaging apparatus 3 transmits an examination start command to the injecting apparatus 2. Then, the injecting apparatus 2, which has received the examination start command, causes the injection head 21 to start the injection based on the determined injection protocol, and transmits an injection start signal to the imaging apparatus 3. The injecting apparatus 2, which has received the examination start command, may calculate the time from the injection start to the imaging start based on the imaging condition acquired from the imaging apparatus 3, and display, for example, an imaging start time or a remaining time until the imaging starts on the head display 211. As a result, the operator can be urged to leave the examination room, thereby protecting him or her from radiation exposure.

The operator can also start the examination by pressing, for example, a start button displayed on the touch panel 26 of the injecting apparatus 2, the hand switch connected to the console 23, or the start button arranged on the injection head 21. As a result, the injecting apparatus 2 transmits the examination start command to the imaging apparatus 3. Then, the injecting apparatus 2 causes the injection head 21 to start the injection based on the determined injection protocol, and transmits the injection start signal to the imaging apparatus 3.

The imaging apparatus 3 and the injecting apparatus 2 then operate in synchronization with each other. For example, when the imaging apparatus 3 has stopped imaging, the imaging apparatus 3 transmits an imaging stop signal to the injecting apparatus 2, and the injecting apparatus 2 stops the injection. Further, when the injection by the injecting apparatus 2 has been aborted, the injecting apparatus 2 transmits an injection stop signal to the imaging apparatus 3. In this case, the imaging apparatus 3 may stop imaging.

When the injection has finished, the injecting apparatus 2 transmits an injection finish signal to the imaging apparatus 3. The imaging apparatus 3, which has received the injection finish signal, causes the imaging unit 31 to start the imaging based on the determined imaging plan, and transmits an imaging start signal to the injecting apparatus 2. However, the timing for starting the imaging is set as appropriate based on the imaging plan. For example, the imaging apparatus 3 can start the imaging before the injection has finished, and transmit the imaging start signal to the injecting apparatus 2. In this case, the imaging apparatus 3, which has received the injection start signal, measures the time from the injection start by a timer included in the imaging apparatus 3, and starts the imaging after a predetermined time has elapsed from the injection started. Further, the injecting apparatus 2, which has received the imaging start signal, may stop the chemical liquid injection or may switch a phase of the chemical liquid injection. The imaging apparatus 3 may transmit an imaging finish signal to the injecting apparatus 2 when the imaging has finished.

After imaging has finished, the imaging apparatus 3 transmits exposure time information to the injecting apparatus 2. Further, the imaging apparatus 3 displays the taken image, the exposure time information, and the like, on the display 33. In addition, the imaging apparatus 3 adds identification information, e.g., the imaging date and time, the patient ID, or the examination ID, to the taken image, and transmits the image to the server 100 as an imaging result. The imaging apparatus 3 may also transmit an imaging result consisting of text data to the server 100.

While the chemical liquid is being injected, the injecting apparatus 2 generates a chemical liquid injection history corresponding to the injection protocol, and stores the generated injection history in the storage unit 24. The injection history includes, for example, text data such as the injection protocol, the patient ID, and the examination ID, and image data such as a time-based graph in which one of the horizontal axis and the vertical axis represents an elapsed time, and the another axis represents the injection rate, the injection pressure, the injection amount, or an integrated injection amount. The injecting apparatus 2, which has received the exposure time information from the imaging apparatus 3, adds the exposure time information to the injection history of the injection head 21, and displays the injection history on the touch panel 26.

The injecting apparatus 2 adds identification information, e.g., the imaging date and time, the patient ID, and the examination ID, to the injection history, and transmits the injection history to the server 100 as an injection result. The imaging result data and the injection result data are stored in the server 100. In this way, the examination, which includes chemical liquid injection and imaging, is finished. As a result, the imaging result and the injection result are stored in the server 100 under a state in which the imaging result and the injection result are associated with each other by the identification information. Whether or not the imaging apparatus 3 transmits imaging result information, e.g., the exposure time information, may be freely set by the operator. In this case, the injecting apparatus 2 does not add the imaging result information to the injection history. The injecting apparatus 2 may also transmit the injection result data to the imaging apparatus 3.

Thus, the storage unit 24 of the injecting apparatus 2 stores the injection result information, and the storage unit 34 of the imaging apparatus 3 stores the imaging result information. The imaging apparatus 3 transmits the imaging result information to the injecting apparatus 2, and the injecting apparatus 2 adds the imaging result information to the injection result information. Then, the injecting apparatus 2 transmits the injection result information, which includes the imaging result information, to the server 100 as the injection result. As a result, because the imaging result information can be added to the injection history, a record of the examination content can be stored more accurately.

According to the imaging system 1 of the first embodiment, the imaging apparatus 3 is configured to determine the imaging plan, and to transmit the determined imaging plan to the injecting apparatus 2. Further, the injecting apparatus 2 is configured to automatically determine the injection protocol, which includes the injection condition in accordance with the imaging condition of the received imaging plan. In addition, the injecting apparatus 2 is capable of acquiring the examination order from the imaging apparatus 3.

Because the information for determining the injection protocol is included in the examination order and the imaging plan, the operator does not need to perform the task of inputting such information into the injecting apparatus 2. Further, the need to verify the imaging plan and the injection protocol while alternately looking at the display unit of the imaging apparatus 3 as well as the display unit of the injecting apparatus 2 is eliminated. As a result, the setting of the optimum injection condition in accordance with the imaging condition can be easily performed. In addition, when determining the injection condition based on the imaging condition, because it is not necessary to perform the task of inputting the required information, erroneous inputs and erroneous operations can be prevented.

Even when the imaging condition has been changed, the injecting apparatus 2 is capable of changing the injection condition based on the changed imaging condition. For example, when the imaging condition, e.g., the imaging time, the tube voltage, the imaging range, the rotating speed, or the helical pitch, is changed, there is a possibility that the injection condition is also changed. According to the imaging system 1 of the first embodiment, the changed imaging condition may be acquired by the injecting apparatus 2 from the imaging apparatus 3. The injecting apparatus 2 is configured to automatically determine the injection protocol, which includes the injection condition in accordance with the changed imaging condition. Further, according to the imaging system 1 of the first embodiment, the injecting apparatus 2 is configured to determine the injection condition such that the injection condition satisfies the chemical liquid injectable condition. As a result, injection of the chemical liquid based on an erroneous injection condition can be reliably prevented.

Further, according to the imaging system 1 of the first embodiment, when an injection condition that does not satisfy the injectable condition has been erroneously input, or when the injection condition has been changed to an injection condition that does not satisfy the injectable condition, the injection condition can be automatically corrected.

In particular, when the injection condition is input from an external device including the imaging apparatus 3, or when the operator changes the injection condition, the injection condition may exceed the capabilities of the injection head 21. In such a case, the imaging may be started despite the fact that the chemical liquid cannot be actually injected based on the set injection condition. Further, the injection condition may not satisfy the injectable condition for the patient. In this case, the injection may be started despite the fact that the chemical liquid should not be injected based on the set injection condition. To prevent such a situation, the operator needs to verify the injection conditions, and input the correct injection condition to the injecting apparatus 2. This task forces the operator to pay close attention, and hence places a heavy burden on the operator. In addition, there are cases in which the operator releases the setting of the injecting apparatus 2 and resets the setting, which is a complex task. According to the imaging system 1 of the first embodiment, such burdens and complex tasks can be avoided.

In the first embodiment, the transmitting, receiving, and the like of the imaging condition, the injection condition, and the chemical liquid information may be performed in a different order. For example, the transmitting and receiving of the imaging condition may be performed after the chemical liquid information has been transmitted and received. Further, the transmitting and receiving of the injection condition and the chemical liquid information may be performed after the imaging condition has been verified. In addition, the imaging apparatus 3 may be configured so that the operator can select whether or not to display the injection protocol on the display unit. The injecting apparatus 2 may be configured so that the operator can select whether or not to display the imaging plan on the display unit.

In the first embodiment, the control program of the injecting apparatus 2 is software configured to cause the control unit 25 (computer) to execute the following processing. Specifically, the control program causes the control unit 25 to determine, when the first imaging condition has been acquired from the imaging plan received by the injecting apparatus 2, the first injection condition of the injection protocol in accordance with the first imaging condition. Further, the control program causes the touch panel 26 to display the first injection condition. The control program then causes the control unit 25 to determine, when the second imaging condition, in which the condition has been changed, has been acquired by the injecting apparatus 2, the second injection condition in accordance with the second imaging condition. Further, the control program causes the touch panel 26 to display the second injection condition. The control program is recorded in an internal or external computer-readable recording medium.

The control program of the imaging apparatus 3 is software configured to cause the control unit 35 (computer) to execute the following processing. Specifically, the control program causes the control unit 35 to transmit, when the imaging apparatus 3 has acquired from an external device an examination order including object information, the acquired examination order to the injecting apparatus 2. The control program also causes the control unit 35 to determine the first imaging condition based on the object information. Further, the control program causes the display 33 to display the imaging plan which includes the first imaging condition. In addition, the control program then causes the control unit 35 to transmit, when the operator has changed the first imaging condition to the second imaging condition, the second imaging condition to the injecting apparatus 2. The control program is recorded in an internal or external computer-readable recording medium.

### Second Embodiment

In the first embodiment, in the case of manually changing the injection condition of the injection protocol, the operator performed this operation via the touch panel 26. In the imaging system according to a second embodiment of the present invention, a difference from the first embodiment is that the operator can verify the injection protocol displayed on the display 33 of the imaging apparatus 3, and change the injection condition via the input unit 36. In the description of the second embodiment, differences from the first embodiment are described. The components described in the first embodiment are denoted by the same reference symbols, and descriptions thereof are therefore omitted. Unless otherwise noted, the components denoted by the same reference symbols each have substantially the same operation and function, and actions and effects thereof are also substantially the same.

Similarly to the first embodiment, the various processes performed by the imaging apparatus 3 in the following description are each controlled by the control unit 35 of the imaging apparatus 3 based on a control program. Similarly, the various processes performed by the injecting apparatus 2 are each controlled by the control unit 25 of the injecting apparatus 2 based on a control program.

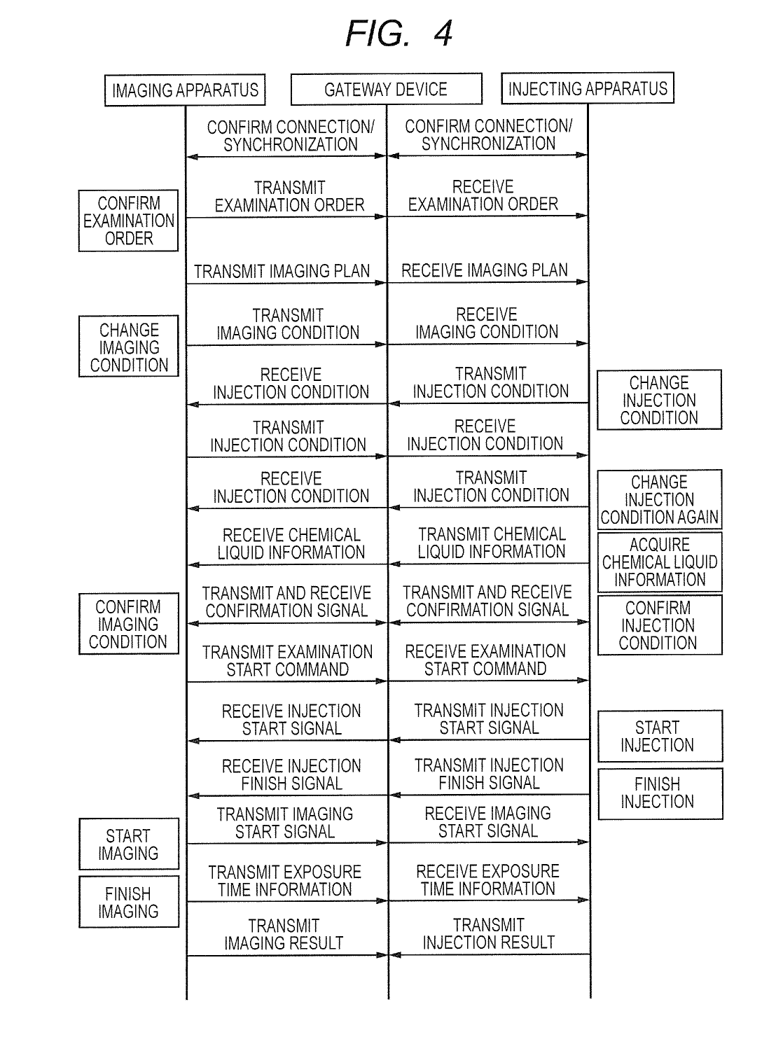

As illustrated in FIG. 4, when the operator turns on the imaging apparatus 3 and the injecting apparatus 2, the imaging apparatus 3 and the injecting apparatus 2 perform a connection/synchronization confirmation with each other. The imaging apparatus 3 acquires an examination order from the server 100, and transmits the examination order, which has been verified by the operator, to the injecting apparatus 2. Then, the injecting apparatus 2 determines the preliminary injection condition of the injection protocol based on, for example, the object information contained in the acquired examination order.

The imaging apparatus 3 determines the first imaging condition of the imaging plan based on, for example, the object information contained in the examination order, and transmits the imaging plan, which has been verified by the operator, to the injecting apparatus 2. Then, the injecting apparatus 2 automatically determines the first injection condition of the injection protocol based on the first imaging condition of the imaging plan, and if necessary changes the preliminary injection condition to the first injection condition.

The imaging apparatus 3 transmits, when the operator has changed the first imaging condition to the second imaging condition, the second imaging condition obtained by the change to the injecting apparatus 2. Then, the injecting apparatus 2 automatically changes the first injection condition of the injection protocol to the second injection condition based on the acquired second imaging condition. The injecting apparatus 2 determines the injection protocol such that the first injection condition and the second injection condition satisfy the chemical liquid injectable condition. The injecting apparatus 2 transmits, when the first injection condition has been determined and when the first injection condition has been changed to the second injection condition, each injection condition to the imaging apparatus 3.

In the second embodiment, the imaging apparatus 3, which has received the first injection condition and the second injection condition, displays the received first injection condition and second injection condition as the injection protocol on the display 33. The operator then verifies the content of the injection protocol by using the display 33 of the imaging apparatus 3. As a result of the verification by the operator, if it is necessary to change the injection condition, e.g., when the state of the patient has changed, the operator manually changes the injection condition from the input unit 36. In this case, the imaging apparatus 3 transmits the changed injection condition to the injecting apparatus 2.

When the injecting apparatus 2 receives the changed injection condition from the imaging apparatus 3, the injecting apparatus 2 causes the safety confirmation unit 251 to compare the changed injection condition and the chemical liquid injectable condition, which is stored in advance in the storage unit 24. In other words, the injecting apparatus 2 includes the safety confirmation unit 251, which determines, when an injection condition has been acquired from an external device, whether or not the acquired injection condition satisfies the injectable condition. The injecting apparatus 2 determines, when the changed injection condition does not satisfy the chemical liquid injectable condition, an injection condition that satisfies the injectable condition, and automatically changes again the acquired injection condition to the determined injection condition. In this case, the injecting apparatus 2 may display the injection condition before re-changing and the injection condition after re-changing on the touch panel 26. Further, the injecting apparatus 2 may display, when the injection condition does not satisfy the injectable condition, a warning on the touch panel 26.

When the injection condition has been changed again, the injecting apparatus 2 transmits the re-changed injection condition to the imaging apparatus 3. Then, the imaging apparatus 3 displays the re-changed injection condition on the display 33. Further, when the injecting apparatus 2 acquires the chemical liquid information, the injecting apparatus 2 transmits the chemical liquid information to the imaging apparatus 3. The imaging apparatus 3, which has received the chemical liquid information, displays the chemical liquid information on the display 33. As a result, the operator can verify the content of the injection protocol and the content of the chemical liquid information by using only the display 33 of the imaging apparatus 3.

When the imaging preparation is complete, the operator then presses the confirmation button of the imaging apparatus 3 in the same manner as in the first embodiment. The imaging apparatus 3 transmits a confirmation signal to the injecting apparatus 2, and waits in a state ready to start the imaging. On the other hand, when the injection preparation is complete, the operator presses the confirmation button of the injecting apparatus 2. As a result, the injecting apparatus 2 transmits a confirmation signal to the injecting apparatus 2, and waits in a state ready to start the injection. When the imaging apparatus 3 receives the confirmation signal from the injecting apparatus 2 and the injecting apparatus 2 receives the confirmation signal from the imaging apparatus 3, the imaging preparation and the injection preparation are complete.

When the operator presses the start button of the imaging apparatus 3, the imaging apparatus 3 transmits an examination start command to the injecting apparatus 2. The injecting apparatus 2, which has received the examination start command, causes the injection head 21 to start the injection based on the determined injection protocol, and transmits an injection start signal to the imaging apparatus 3. Then, when the injection has finished, the injecting apparatus 2 transmits an injection finish signal to the imaging apparatus 3.

Further, the imaging apparatus 3 causes the imaging unit 31 to start to take images based on the determined imaging plan, and transmits an imaging start signal to the injecting apparatus 2. When the imaging has finished, the imaging apparatus 3 transmits the exposure time information to the injecting apparatus 2. The imaging apparatus 3 transmits the imaging result to the server 100, and the injecting apparatus 2 transmits the injection result to the server 100. Based on this, the imaging result data and the injection result data are stored in the server 100.

According to the imaging system 1 of the second embodiment, even when the injection condition of the injection protocol has been changed by the imaging apparatus 3, the safety of the injection condition can be confirmed by the injecting apparatus 2. As a result, injection of the chemical liquid based on an erroneous injection condition can be reliably prevented. Thus, burdens and complex tasks of the operator can be avoided.

According to the imaging system 1 of the second embodiment, similarly to the first embodiment, the operator does not need to perform the task of inputting the information for determining the injection protocol into the injecting apparatus 2. Further, the need to confirm the imaging plan and the injection protocol while alternately looking at the display unit of the imaging apparatus 3 as well as the display unit of the injecting apparatus 2 is eliminated. As a result, the setting of the optimum injection condition in accordance with the imaging condition can be easily performed. In addition, when determining the injection condition based on the imaging condition, because it is not necessary to perform the task of inputting the required information, erroneous inputs and erroneous operations can be prevented.

In the second embodiment, the control program of the injecting apparatus 2 is software configured to cause the control unit 25 (computer) to execute the following processing. In other words, the control program causes, when an injection condition has been acquired from an external device by the injecting apparatus 2, the safety confirmation unit 251 to determine whether or not the acquired injection condition satisfies the injectable condition. When it is determined by the safety confirmation unit 251 that the injectable condition is not satisfied, the control program causes the control unit 25 to determine an injection condition that satisfies the injectable condition, and to automatically change the acquired injection condition to the determined injection condition. Then, the control program causes the control unit 25 to transmit the changed injection condition to the imaging apparatus 3.

The control program of the imaging apparatus 3 is software configured to cause the control unit 35 (computer) to execute the following processing. In other words, when the injection condition has been changed at the imaging apparatus 3, the control program causes the control unit 35 to transmit the changed injection condition to the injecting apparatus 2. When the automatically changed injection condition has been received from the injecting apparatus 2, the control program causes the display 33 to display the received injection condition.

The injecting apparatus 2 of the second embodiment may be configured so that the operator can select between a mode in which the injection condition is changed again and a mode in which re-changing the injection condition is omitted.

The present invention is described above referring to the embodiments. However, the present invention is not limited to the above-mentioned embodiments. The present invention also encompasses the invention modified within a scope not deviated from the present invention, and the invention equivalent to the present invention. Further, the above-mentioned embodiments and modifications may be combined with each other as appropriate within the scope not deviated from the present invention.

For example, the gateway device 110 may be omitted, and the injecting apparatus 2 may be directly connected to the imaging apparatus 3 by a cable or wirelessly. Further, the gateway device 110 may be constructed integrally with the injecting apparatus 2 or the imaging apparatus 3. In addition, the injecting apparatus 2 may acquire the taken image from the imaging apparatus 3 or the server 100, and display the acquired image and the injection history on the touch panel 26. Similarly, the imaging apparatus 3 may acquire the injection history from the injecting apparatus 2 or the server 100, and display the taken image and the injection history on the display 33. Further, the injecting apparatus 2 or the imaging apparatus 3 may generate an image that combines the taken image and the injection history, and store the generated combined image in the server 100. The injecting apparatus 2 may also acquire from the server 100 information relating to the examination order, for example.

The imaging apparatus 3 may acquire the injection condition from the injection protocol received from the injecting apparatus 2, and automatically change the imaging condition of the imaging plan based on the acquired injection condition. Further, the injecting apparatus 2 may select, when determining the injection protocol in accordance with the imaging plan, one injection protocol from among a plurality of injection protocols stored in the storage unit 24. In addition, the injecting apparatus 2 may select, when information relating to the injection protocol used during the previous imaging is included in the acquired examination order, that injection protocol. Even in this case, the injecting apparatus 2 may change the injection condition of the injection protocol based on the imaging condition of the imaging plan received from the imaging apparatus 3.

In the case of performing an examination on an urgent patient, when the examination order is not stored in the server 100, the operator can directly input the necessary information into the imaging apparatus 3 and the injecting apparatus 2. In this case, the imaging apparatus 3 determines the imaging plan based on the input information, and the injecting apparatus 2 also determines the injection protocol based on the input information. Even in this case, the injecting apparatus 2 is capable of changing the injection condition of the injection protocol based on the imaging condition of the imaging plan received from the imaging apparatus 3. Further, the operator can verify the injection protocol, the imaging plan, or both the injection protocol and the imaging plan, by using the touch panel 26 or the head display 211. In addition, when necessary, the operator can manually change the injection protocol, the imaging plan, or both the injection protocol and the imaging plan. Still further, a stop button may be arranged on the operation unit 212 of the injection head 21. As a result, when there is a problem with the patient, the injecting apparatus 2, or the imaging apparatus 3 during the period from injection start to exposure (imaging) start, the operator can stop the injection, thus ensuring safety.

The imaging apparatus 3, which has received the chemical liquid information from the injecting apparatus 2, may change the imaging condition of the imaging plan. For example, the imaging apparatus 3 may automatically change, when chemical liquid information relating to the concentration of the contrast medium has been received, the imaging condition such that the tube voltage increases or decreases. Further, the imaging apparatus 3 may display on the display 33 an imaging condition that is recommended based on the chemical liquid information. In addition, the injecting apparatus 2 may transmit to the imaging apparatus 3 the imaging condition that is recommended based on the chemical liquid information. In this case, the imaging apparatus 3 changes the imaging plan so as to include the received imaging condition, or displays the received imaging condition on the display 33. Still further, the injecting apparatus 2 or the imaging apparatus 3 may transmit the chemical liquid information to an external storage device, e.g., the server 100. In this case, the external storage device stores the received chemical liquid information. For example, when an external storage device included in an accounting system stores chemical liquid information relating to a type of the contrast medium, accounting of the contrast medium can be processed accurately, and requests for an erroneous monetary amount due to manual input can be prevented.

The control unit 35 of the imaging apparatus 3 may also be configured to determine a unique injection protocol (unique injection condition). In this case, the control unit 35 is configured to compare the injection protocol (first injection condition or second injection condition) received from the injecting apparatus 2 and the unique injection condition. When the first injection condition or the second injection condition does not match the unique injection condition, the control unit 35 notifies the operator by issuing a mismatch notification. For example, as the mismatch notification, an indication representing a mismatch or a sound representing a mismatch is issued. The control unit 35 is configured to perform the injection condition comparison when the confirmation button displayed on the display 33 is pressed. The control unit 35 may also be configured to perform the injection condition comparison when transmitting the confirmation signal (FIG. 3 and FIG. 4), when the first injection condition or the second injection condition is received, or when the confirmation signal is received.

The operator, who has received the mismatch notification, selects any one of the injection condition received from the injecting apparatus 2 and the unique injection condition. The control unit 35 is configured to then replace, when the injection condition received from the injecting apparatus 2 is selected, the unique injection condition with the selected injection condition, and to transmit a confirmation signal to the injecting apparatus 2. On the other hand, the control unit 35 is configured to transmit, when the unique injection condition is selected, the selected injection condition to the injecting apparatus 2. The control unit 25 of the injecting apparatus 2 is configured to replace the determined injection condition with the selected injection condition, and to transmit a confirmation signal to the imaging apparatus 3. Further, the injecting apparatus 2 may be configured to display, when the selected injection condition does not satisfy the injectable condition, a warning on the touch panel 26.

The control unit 35 of the imaging apparatus 3 is configured to determine that there is a mismatch when at least one of the injection amount, the injection rate, the injection time, or the injection pressure is different, or when an injection condition other than those conditions is present in the injection protocol received from the injecting apparatus 2. The mismatch notification displayed on the display 33 may include, other than a notification indicating a mismatch, for example, a mismatching item, each of the injection conditions, and the like. The imaging system 1 including such an imaging apparatus 3 enables an injection condition that is not desired by the operator to be changed. As a result, the injection of the chemical liquid based on an erroneous injection condition can be reliably prevented.

The control unit 25 of the injecting apparatus 2 may be configured to compare the unique injection condition determined by the imaging apparatus 3 and the injection condition (first injection condition or second injection condition) determined by the injecting apparatus 2, and to issue a mismatch notification when the two compared injection conditions do not match. Further, the imaging apparatus 3 may be configured to perform comparison with use of an injection condition input by the operator as the unique injection condition.

### Modified Embodiment

The imaging apparatus 3 may also be configured to acquire from an external device, e.g., the injecting apparatus 2, another injection condition (third injection condition). In this case, the control unit 35 of the imaging apparatus 3 is configured to compare the injection condition (first injection condition or second injection condition) received from the injecting apparatus 2 and the third injection condition. Further, the control unit 35 is configured to notify, when the first injection condition or the second injection condition does not match the third injection condition, the operator by issuing a mismatch notification.

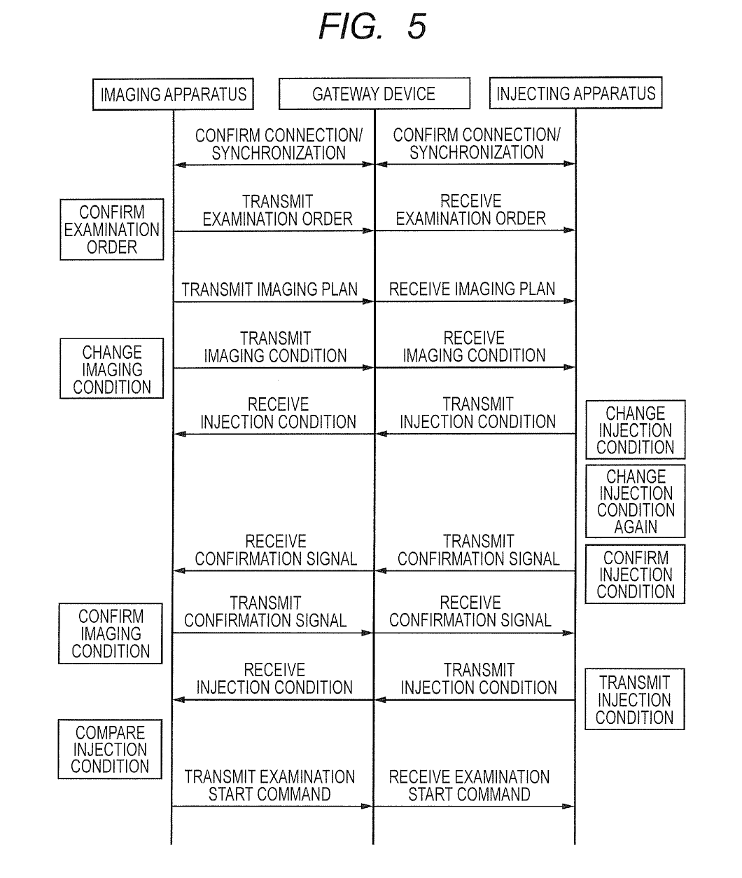

An imaging system according to this modified embodiment is described with reference to FIG. 5. The various processes performed by the imaging apparatus 3 in the following description are each controlled by the control unit 35 of the imaging apparatus 3 based on a control program. Similarly, the various processes performed by the injecting apparatus 2 are each controlled by the control unit 25 of the injecting apparatus 2 based on a control program.

As illustrated in FIG. 5, after performing a connection/synchronization confirmation, the imaging apparatus 3 transmits to the injecting apparatus 2 the examination order verified by the operator. Then, the injecting apparatus 2 determines the preliminary injection condition of the injection protocol based on, for example, the object information contained in the acquired examination order. The imaging apparatus 3 then determines the first imaging condition of the imaging plan based on, for example, the object information contained in the examination order, and transmits to the injecting apparatus 2 the imaging plan verified by the operator. Then, the injecting apparatus 2 determines the first injection condition of the injection protocol based on the first imaging condition of the imaging plan, and changes the preliminary injection condition to the first injection condition as necessary.

When the operator has changed the first imaging condition to the second imaging condition, the imaging apparatus 3 transmits the second imaging condition obtained by the change to the injecting apparatus 2. Then, the injecting apparatus 2 changes the first injection condition to the second injection condition (“change injection condition” in FIG. 5) based on the acquired second imaging condition. The injecting apparatus 2 transmits, automatically or in response to a request from the imaging apparatus 3, the first injection condition and the second injection condition to the imaging apparatus 3.

Then, when a change in the injection condition is necessary, the operator changes again the injection condition manually via the touch panel 26 of the injecting apparatus 2 (“change injection condition again” in FIG. 5). When the injection preparation is complete, the operator presses the confirmation button of the injecting apparatus 2. As a result, the injecting apparatus 2 transmits a confirmation signal to the injecting apparatus 2, and waits in a state ready to start the injection. In the embodiment illustrated in FIG. 5, when the injection condition is changed again, the injecting apparatus 2 does not transmit the injection condition to the imaging apparatus 3.

Then, when the imaging preparation is complete, the operator presses the confirmation button of the imaging apparatus 3. The imaging apparatus 3 then transmits a confirmation signal to the injecting apparatus 2, and waits in a state ready to start the imaging. In this case, the injecting apparatus 2 transmits another injection protocol that has been changed again (third injection condition) to the imaging apparatus 3 (“transmit injection condition” in FIG. 5). Then, the control unit 35 of the imaging apparatus 3 compares the third injection condition and the first injection condition or the second injection condition received in advance (“compare injection condition” in FIG. 5).

When it is determined as a result of the comparison that the first injection condition or the second injection condition and the third injection condition do not match, the control unit 35 notifies the operator by issuing a mismatch notification. Then, the operator, who has received the mismatch notification, selects any one of the first injection condition or the second injection condition received in advance and the third injection condition. The control unit 35 replaces, when the third injection condition is selected, the first injection condition or the second injection condition received in advance with the selected third injection condition, and again transmits a confirmation signal to the injecting apparatus 2. On the other hand, the control unit 35 transmits, when the first injection condition or the second injection condition received in advance is selected, the selected injection condition to the injecting apparatus 2. Then, the control unit 25 of the injecting apparatus 2 replaces the third injection condition with the received injection condition, and again transmits a confirmation signal to the imaging apparatus 3.

When the imaging apparatus 3 receives the confirmation signal from the injecting apparatus 2 and the injecting apparatus 2 receives the confirmation signal from the imaging apparatus 3, the imaging preparation and the injection preparation are complete. Then, when the operator presses the start button of the imaging apparatus 3, the imaging apparatus 3 transmits an examination start command to the injecting apparatus 2. Because the subsequent processing flow is the same as in the first embodiment and the second embodiment, a description thereof is omitted here.

According to the imaging system 1 of such a modified embodiment, injection of the chemical liquid based on an erroneous injection condition can be reliably prevented. Further, the imaging apparatus 3 may acquire the third injection condition from the server 100 or an external storage medium. The third injection condition may also be input by the operator into the imaging apparatus 3. In addition, transmitting and receiving of the third injection condition may be performed at a timing different from that described above. For example, the injecting apparatus 2 may be configured to transmit the third injection condition together with the confirmation signal. Further, the injecting apparatus 2 may be configured to change the injection condition again and then subsequently transmit the third injection condition. The injection condition comparison may be performed any time after the third injection condition has been acquired.

The present application claims the benefit of priority from Japanese Patent Application No. 2014-009094, filed on Jan. 22, 2014, the content of which is incorporated herein by reference.

### REFERENCE SIGNS LIST

1: imaging system, 2: injecting apparatus, 3: imaging apparatus, 20: communication unit, 21: injection head, 22: stand, 23: console, 24: storage unit, 25: control unit, 26: touch panel, 30: communication unit, 31: imaging unit, 32: control device, 33: display, 34: storage unit, 35: control unit, 36: input unit, 100: server, 110: gateway device, 211: head display, 212: operation unit, 251: safety confirmation unit

1. An imaging system, comprising an injecting apparatus for a chemical liquid and an imaging apparatus for medical use, which is connectable to the injecting apparatus and which takes an image of an object,
the imaging apparatus comprising:
a control unit configured to determine a first imaging condition based on externally acquired object information, and to transmit the first imaging condition to the injecting apparatus; and
an input unit configured to input a second imaging condition, which is changed from the first imaging condition,
the injecting apparatus comprising:
a control unit configured to determine a first injection condition based on the first imaging condition, and to change, when the second imaging condition is received from the imaging apparatus, the first injection condition to a second injection condition based on the second imaging condition.

2. An imaging system according to claim 1,
wherein the control unit of the imaging apparatus is configured to transmit the object information to the injecting apparatus before transmission of the first imaging condition, and
wherein the control unit of the injecting apparatus is configured to determine a preliminary injection condition based on the object information, and to change, when the first imaging condition is received, the preliminary injection condition to the first injection condition based on the first imaging condition.

3. An imaging system according to claim 1, wherein the control unit of the injecting apparatus is configured to determine the first injection condition and the second injection condition such that the first injection condition and the second injection condition satisfy an injectable condition.

4. An imaging system according to claim 1, wherein the injecting apparatus further comprises a safety confirmation unit configured to determine, when externally acquiring an injection condition, whether or not the acquired injection condition satisfies an injectable condition.

5. An imaging system according to claim 1,
wherein the injecting apparatus further comprises a storage unit configured to store injection result information,
wherein the control unit of the imaging apparatus is configured to transmit imaging result information to the injecting apparatus, and
wherein the control unit of the injecting apparatus is configured to add the imaging result information to the injection result information.

6. An imaging system according to claim 1,
wherein the injecting apparatus further comprises a display unit which displays the first injection condition, and an injection unit which injects the chemical liquid and comprises a display, and
wherein the control unit of the injecting apparatus is configured to display the first imaging condition on the display unit and on the display.

7. An imaging system according to claim 1, wherein the control unit of the imaging apparatus is configured to:
determine a unique injection condition;
compare the first injection condition or the second injection condition with the unique injection condition; and
perform a mismatch notification when the first injection condition or the second injection condition mismatches with the unique injection condition.

8. An imaging system, comprising an injecting apparatus for a chemical liquid and an imaging apparatus for medical use, which is connectable to the injecting apparatus and which takes an image of an object,
the imaging apparatus comprising:
a control unit configured to determine a first imaging condition based on externally acquired object information, and to transmit the first imaging condition to the injecting apparatus; and
an input unit configured to input a second imaging condition, which is changed from the first imaging condition,
the injecting apparatus comprising:
a control unit configured to determine a first injection condition based on the first imaging condition, and to change, when the second imaging condition is received from the imaging apparatus, the first injection condition to a second injection condition based on the second imaging condition,
wherein the control unit of the imaging apparatus is configured to:
acquire a third injection condition, externally;
compare the first injection condition or the second injection condition with the third injection condition; and
perform a mismatch notification when the first injection condition or the second injection condition mismatches with the third injection condition.

9. An injecting apparatus for a chemical liquid, which is connectable to an imaging apparatus for medical use, which takes an image of an object, the injecting apparatus comprising:
a control unit configured to determine a first injection condition based on a first imaging condition received from the imaging apparatus, and to change, when a second imaging condition which is changed from the first imaging condition is received from the imaging apparatus, the first injection condition to a second injection condition based on the second imaging condition.
